SEQUENCE LISTING (16 sequences)

SEQ ID NO: 1 - DNA, 21 bp, Homo sapiens
aagtccaatacctcactcgct

SEQ ID NO: 2 - DNA, 21 bp, Homo sapiens
aagcacgtcaaaagctacaga

SEQ ID NO: 3 - DNA, 19 bp, Homo sapiens
aatttctgtctcatcttaa

SEQ ID NO: 4 - DNA, 19 bp, Homo sapiens
ggtcttcaccaagtatcaa

SEQ ID NO: 5 - DNA, 19 bp, Homo sapiens
ggccatactcttacataat

SEQ ID NO: 6 - DNA, 19 bp, Homo sapiens
ggcaaggaaaataaaagat

SEQ ID NO: 7 - DNA, 19 bp, Homo sapiens
gcacgtcaaaagctacaga

SEQ ID NO: 8 - DNA, 19 bp, Homo sapiens
ggcactgtagtgaattatc

SEQ ID NO: 9 - DNA, 19 bp, Homo sapiens
gctagagttacctagctta

SEQ ID NO: 10 - DNA, 21 bp, Mus musculus
aaagtgcaatacctcactcgc

SEQ ID NO: 11 - DNA, 21 bp, Mus musculus
aatttctgcctcatcttgata

SEQ ID NO: 12 - DNA, 21 bp, Rattus norvegicus
aaagtgcaataccttactcgc

SEQ ID NO: 13 - DNA, 21 bp, Rattus norvegicus
aatttctgtctcatcttgata

SEQ ID NO: 14 - DNA, 29 bp, Artificial
  siRNA Sense Oligonucleotide
aaagtccaataccttactcgccctgtctc

SEQ ID NO: 15 - DNA, 29 bp, Artificial
  siRNA Antisense Oligonucleotide
aagcgagtaaggtattggactcctgtctc

SEQ ID NO: 16 - DNA, 8 bp, Artificial
  3′ End Tail of siRNA Sense and Antisense oligonucleotides
cctgtctc

(19) United States
(12) Patent Application Publication
(10) Pub. No.: US 2006/0198825 A1
(43) Pub. Date: Sep. 7, 2006

(54) Reagents, methods and systems to suppress phospholamban expression

(76) Inventors: William F. Kaemmerer, Edina, MN (US); Shannon M. Larkin, Burnsville, MN (US); Orhan Soykan, Shoreview, MN (US); Timothy H. Robinson, Savage, MN (US)

Correspondence Address: FOX ROTHSCHILD LLP;PRINCETON PIKE CORPORATE CENTER, 997 LENOX DRIVE, BUILDING #3, LAWRENCEVILLE, NJ, 08648, US

(21) Appl. No.: 11/071,609

(22) Filed: Mar. 3, 2005

(51) Int. Cl. A61K 48/00; C07H 21/02; A61M 31/00

(52) U.S. Cl. 424/93.2; 514/44; 536/23.1; 604/500

(57) ABSTRACT

The present invention relates to reagents, methods and systems to treat heart failure using small interfering RNA (siRNA) molecules targeted to phospholamban.

### FIELD OF THE INVENTION

The present invention relates to treatments for heart failure using small interfering RNA (siRNA) targeted to phospholamban.

### BACKGROUND OF THE INVENTION

Cardiac disease leading to heart failure is the leading cause of combined morbidity and mortality in the developed world. Heart failure is a complex clinical syndrome in which the heart is incapable of maintaining a cardiac output adequate to accommodate metabolic demand and the venous return of blood to the heart. Worldwide, 22 million people are living with this disease, and 5 million of them are in the United States. Two million new cases are diagnosed annually, of which 500,000 are in the United States. It is estimated that heart failure afflicts about 1 percent of people over the age of 65 in the United States.

Heart failure is caused by the loss of a critical quantity of properly functioning myocardial cells after injury to the heart due to one or more of the following: ischemic heart disease, hypertension, ideopathic cardiomyopathy, pathogenic infections (e.g., viral myocarditis, Chagas' disease), toxins (e.g., alcohol or cytotoxic drugs), valvular disease, and prolonged arrhythmias.

Heart failure results in a marked decrease in the contractility and relaxation of the cardiac muscle, resulting in reduced cardiac output and increased blood pressure in the venous system. Left ventricular dysfunction associated with heart failure manifests itself in various systolic and diastolic symptoms, including impaired systolic contractility and ejection and impaired diastolic filling and relaxation. Approximately two-thirds of heart failure subjects have systolic dysfunction. In both systolic and diastolic impairment, mechanical dysfunctions of the cardiomyocytes affect the hemodynamic properties of the heart.

Many traditional treatments are aimed at the management of the hemodynamic symptoms of pump dysfunction and the biochemical abnormalities that depress contractility and slow relaxation in cardiac myocytes. Diuretics are often used to reduce pulmonary edema and dyspnea in subjects with fluid overload and are usually used in conjunction with angiotensin converting enzyme (ACE) inhibitors for vasodilation. Digoxin is another popular choice for treating cardiac disease as an ionotropic agent; however, doubts remain concerning the long-term efficacy and safety of digoxin. Carvedilol, a β-blocker, has been introduced to complement the above treatments in order to slow down the progression of cardiac disease. Antiarrhythmic agents can be used in order to reduce the risk of sudden death in subjects suffering from cardiac disease. Lastly, heart transplants have been effective in the treatment of subjects with advanced stages of cardiac disease; however, the limited supply of donor hearts greatly limits the scope of this treatment to the broad population.

While the above treatment strategies can all improve morbidity and mortality associated with cardiac disease, the only existing definitive approach to curing the diseased heart is replacement by transplant. Even a healthy, transplanted heart can become diseased in response to the various stresses of mechanical, hemodynamic, hormonal, and pathological stimuli associated with extrinsic risk factors. As such, there exists the need for therapeutics directed to the underlying biological pathways involved in the initiation and progression of cardiac muscle dysfunction.

One pathway that holds promise as a therapeutic target for heart failure is the cardiomyocyte calcium regulation pathway. Abnormal calcium compartmentalization is a reproducible feature of heart failure.

The sarcoplasmic reticulum (SR), which makes up about 0.1% of the cardiomyocyte's volume, stores, releases and re-sequesters most of the calcium responsible for the contraction and relaxation of the cardiomyocyte. Calcium in the cell exists as a cation (Ca²⁺). Release and re-sequestration of Ca²⁺ in the SR occurs as part of each cardiac cycle.

The cycle begins with an initial flux of Ca²⁺ into the cardiomyocyte, arising from a chemical or electrical triggering event. Chemically, the release of activating neurotransmitters from the sympathetic nervous system regulates the entry of extracellular calcium into the cell through norepinephrine or epinephrine-sensitive Ca²⁺ ion channels. Once the triggering Ca²⁺ enters the cell, it binds to ryanodine receptors on the sarcoplasmic reticulum, initiating release of stored calcium ions from the SR.

Electrically, the initial flux of calcium arises in response to a change in the voltage difference across the cardiomyocyte's membrane. Voltage sensitive Ca²⁺ ion channels in the membrane open in response to the change in the voltage difference across the membrane, resulting in an inward flux of Ca²⁺. T-tubules, which are invaginations of the outer cell membrane, ensure that these ion channels, and the inward calcium flux they produce in response to electrical depolarization, are in close proximity to the SR.

As a result of the inward flux of Ca²⁺ arising from these triggers, massive amounts of calcium ions are released by the sarcoplasmic reticulum into each sarcomere within the myofibrils. The free Ca²⁺ binds to troponin, causing the tropinin-tropomyosin strands to generate contractile force. This period of the cardiac cycle is known as systole.

During relaxation, Ca²⁺ is taken up by the sarcoplasmic reticulum, and stored until the onset of the next contraction. As the free Ca²⁺ in the cytosol is released from troponin and sequestered back in the SR, the troponin-tropomyosin strands that line the actin filaments shift back towards the outside of the actin filament, and the myosin heads are released from the actin. As a result, the filaments in each sarcomere slide back to their resting position, and the cell relaxes. This period of the cardiac cycle is known as diastole.

Release of Ca²⁺ from the SR is achieved using the concentration gradient as the driving force. However, re-sequestration of Ca²⁺ back into the SR must be done against the concentration gradient, which requires a molecular pump known as the SR Ca²⁺-ATPase 2 (SERCA2).

SERCA2 is a calcium ATPase that resides in the membranes of the SR. A central pore formed by SERCA2 in the membrane selectively conducts calcium ions, using energy derived from ATP to move these ions into the SR against the ion concentration gradient. Decreased activity of SERCA2 results in inappropriate handling of Ca²⁺ in the heart. If the function of SERCA2 is inadequate, not all the cytoplasmic free Ca²⁺ is sequestered back into the SR. The continued presence of some free Ca²⁺ in the sarcomeres prevents the complete relaxation of the heart, which manifests itself as diastolic heart failure. Because the loading of the SR with free Ca²⁺ is incomplete, a lesser amount is discharged during the next cardiac cycle, causing a weaker contraction, which manifests as systolic dysfunction.

SERCA2 activity is regulated by phospholamban, a 52 amino acid muscle-specific SR phosphoprotein. In its active, unphosphorylated state, phospholamban is a potent inhibitor of SERCA2 affinity for Ca²⁺. Phosphorylation of phospholamban at serine 16 or threonine 17 by cyclic AMP-dependent protein kinase (PKA) or calmodulin kinase results in the inhibition of phospholamban interaction with SERCA2. This phosphorylation event is predominantly responsible for the proportional increase in the rate of Ca²⁺ uptake into the SR and resultant ventricular relaxation.

Because heart failure is intimately associated with abnormal Ca²⁺ compartmentalization resulting from impaired Ca²⁺ uptake in the SR by SERCA2, increasing the phosphorylation of phospholamban or the ratio of SERCA2 to phospholamban would be expected to restore cardiomyocyte function and thus have utility as treatments for heart failure. Indeed, several animal studies have demonstrated this to be the case. For example, inhibition of SERCA2-phospholamban interaction via in vivo expression of a phospholamban point mutant dominantly activated the contractility of ventricular muscle cells and rescued the spectrum of phenotypes in a mouse model for human heart failure (Minamisawa et al., Cell 99:313 (1999). In addition, adeno-associated viral gene transfer of a pseudophosphorylated mutant of human phospholamban in cardiomyopathic hamsters enhanced myocardial SR Ca²⁺ uptake and suppressed progressive impairment of left ventricular systolic function and contractility for 28-30 weeks (Hoshijima et al., Nat. Med. 8:864 (2002).

Studies with human cells have also demonstrated the feasibility of restoring cardiomyocyte function by targeting the SERCA2/phospholamban pathway. For example, adenoviral gene transfer of SERCA2 restored contractile function and calcium handling in isolated cardiomyocytes from failing human hearts (del Monte et al., Circulation 100:2308 (1999). Similarly, adenoviral-mediated expression of phospholamban antisense RNA restored contractility in failing human ventricular cells (del Monte et al., Circulation 105:904 (2002).

While antisense appears to be a viable method of regulating phospholamban levels in cardiomyocytes, large excess of antisense nucleotides is required to achieve the desired effect. In addition, expression of dominant wild-type and mutant proteins has proved useful only to a limited degree because of the variability (both in levels and tissue distribution) of protein expression. Accordingly, there is an immediate need for improved reagents, methods and systems for treating heart failure through the regulation of levels of phospholamban in cardiomyocytes.

### SUMMARY OF THE INVENTION

The present invention fills the foregoing need by providing reagents, methods and systems for regulating cellular levels of phospholamban. Applicants have found that small interfering RNA (siRNA) molecules that correspond to at least a portion of a phospholamban nucleic acid sequence are effective in inhibiting the expression of phospholamban, thereby providing a means for treating heart failure. Upon the inhibition of phospholamban expression, SERCA2 function is enhanced, resulting in increased calcium uptake within cardiomyocytes and improved cardiovascular hemodynamics.

Accordingly, one aspect of the present invention is directed to a siRNA molecule corresponding to at least a portion of a phospholamban nucleic acid sequence capable of inhibiting expression of phospholamban in a cell.

Another aspect of the present invention is directed to an expression vector comprising at least one DNA sequence encoding a siRNA molecule corresponding to at least a portion of a phospholamban nucleic acid sequence capable of inhibiting expression of phospholamban in a cell operably linked to a genetic control element capable of directing expression of said siRNA molecule in a host cell.

Another aspect of the present invention is directed to a method for inhibiting expression of phospholamban in a heart cell comprising introducing into said heart cell at least one siRNA molecule that corresponds to at least a portion of a phospholamban nucleic acid sequence.

Another aspect of the present invention is directed to a method for treating a subject suffering from heart failure comprising introducing into said subject at least one siRNA molecule that corresponds to at least a portion of a phospholamban nucleic acid sequence.

Another aspect of the present invention is directed to a system for treating a patient suffering from heart failure comprising at least one siRNA molecule that corresponds to at least a portion of a phospholamban nucleic acid sequence and a means for introducing said siRNA molecule to the heart of the patient.

### BRIEF DESCRIPTION OF THE DRAWINGS

FIG. 1 shows a detailed view of a dual catheter delivery system for delivery of phospholamban siRNA molecules to cardiac muscle.

### DETAILED DESCRIPTION

To aid in the understanding of the invention, the following non-limiting definitions are provided:

The term “gene” refers to a DNA sequence that comprises control and coding sequences necessary for the production of a polypeptide or its precursor. The polypeptide can be encoded by a full length coding sequence (either genomic DNA or cDNA) or by any portion of the coding sequence so long as the desired activity is retained. In some aspects, the term “gene” also refers to an mRNA sequence or a portion thereof that directly codes for a polypeptide or its precursor.

The term “transfection” refers to the uptake of foreign DNA by a cell. A cell has been “transfected” when exogenous (i.e., foreign) DNA has been introduced inside the cell membrane. Transfection can be either transient (i.e., the introduced DNA remains extrachromosomal and is diluted out during cell division) or stable (i.e., the introduced DNA integrates into the cell genome or is maintained as a stable episomal element).

“Cotransfection” refers to the simultaneous or sequential transfection of two or more vectors into a given cell.

The term “promoter element” or “promoter” refers to a DNA regulatory region capable of binding an RNA polymerase in a cell (e.g., directly or through other promoter-bound proteins or substances) and initiating transcription of a coding sequence. A promoter sequence is, in general, bounded at its 3′ terminus by the transcription initiation site and extends upstream (5′ direction) to include the minimum number of bases or elements necessary to initiate transcription at any level. Within the promoter sequence may be found a transcription initiation site (conveniently defined, for example, by mapping with nuclease S1), as well as protein binding domains (consensus sequences) responsible for the binding of RNA polymerase. The promoter may be operably associated with other expression control sequences, including enhancer and repressor sequences.

The term “in operable combination”, “in operable order” or “operably linked” refers to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

The term “vector” refers to a nucleic acid assembly capable of transferring gene sequences to target cells (e.g., viral vectors, non-viral vectors, particulate carriers, and liposomes). The term “expression vector” refers to a nucleic acid assembly containing a promoter which is capable of directing the expression of a sequence or gene of interest in a cell. Vectors typically contain nucleic acid sequences encoding selectable markers for selection of cells that have been transfected by the vector. Generally, “vector construct,” “expression vector,” and “gene transfer vector,” refer to any nucleic acid construct capable of directing the expression of a gene of interest and which can transfer gene sequences to target cells. Thus, the term includes cloning and expression vehicles, as well as viral vectors.

The term “antibody” refers to a whole antibody, both polyclonal and monoclonal, or a fragment thereof, for example a F(ab)₂, Fab, FV, VH or VK fragment, a single chain antibody, a multimeric monospecific antibody or fragment thereof, or a bi- or multi-specific antibody or fragment thereof. The term also includes humanized and chimeric antibodies.

The term “heart failure” includes congestive heart failure, heart failure with diastolic dysfunction, heart failure with systolic dysfunction, heart failure associated with cardiac hypertrophy, and heart failure that develops as a result of chemically induced cardiomyopathy, congenital cardiomyopathy, and cardiomyopathy associated with ischemic heart disease or myocardial infarction.

The term “treating” or “treatment” of a disease refers to executing a protocol, which may include administering one or more drugs to a patient (human or otherwise), in an effort to alleviate signs or symptoms of the disease. Alleviation can occur prior to signs or symptoms of the disease appearing, as well as after their appearance. Thus, “treating” or “treatment” includes “preventing” or “prevention” of disease. In addition, “treating” or “treatment” does not require complete alleviation of signs or symptoms, does not require a cure, and specifically includes protocols which have only a marginal effect on the patient.

The term “patient” refers to a biological system to which a treatment can be administered. A biological system can include, for example, an individual cell, a set of cells (e.g., a cell culture), an organ, a tissue, or a multi-cellular organism. A patient can refer to a human patient or a non-human patient.

Aspects of the present invention provides reagents, methods and systems for inhibiting expression of phospholamban in a heart cell using siRNA molecules that correspond to at least a portion of a phospholamban nucleic acid sequence. Applicants have found that siRNA molecules targeted to phospholamban mRNA are effective in inhibiting expression of phospholamban, thereby providing improved methods for treating heart failure. The methods of the present invention can be performed utilizing routine techniques in the field of molecular biology. Basic texts disclosing general molecular biology methods include Sambrook et al., Molecular Cloning, A Laboratory Manual (3d ed. 2001) and Ausubel et al., Current Protocols in Molecular Biology (1994). More specialized texts relevant to the present invention include Sohail, Gene Silencing by RNA Interference: Technology and Application (2004).

One aspect of the present invention provides a siRNA molecule corresponding to at least a portion of a phospholamban nucleic acid sequence capable of inhibiting expression of phospholamban in a cell. siRNAs are typically short (19-29 nucleotides), double-stranded RNA molecules that cause sequence-specific degradation of complementary target mRNA known as RNA interference (RNAi) (Bass, Nature 411:428 (2001)). Accordingly, in some embodiments, the siRNA molecules comprise a double-stranded structure comprising a sense strand and an antisense strand, wherein the antisense strand comprises a nucleotide sequence that is complementary to at least a portion of a phospholamban nucleic acid sequence and the sense strand comprises a nucleotide sequence that is complementary to at least a portion of the nucleotide sequence of said antisense region, and wherein the sense strand and the antisense strand each comprise about 19-29 nucleotides.

Any phospholamban nucleic acid sequence can be targeted by the siRNA molecules of the present invention. Nucleic acid sequences encoding phospholamban from various species are publicly available from Genbank and include human (NM_{—}002667), mouse (NM_{—}023129), rat (NM_{—}022707), chicken (NM_{—}205410), dog (NM_{—}001003332), pig (NM_{—}214213), and rabbit (Y00761). Preferably, the targeted phospholamban nucleic acid sequence is mammalian, more preferably human.

The siRNA molecules targeted to phospholamban can be designed based on criteria well known in the art (e.g., Elbashir et al., EMBO J. 20:6877 (2001)). For example, the target segment of the target mRNA preferably should begin with AA (most preferred), TA, GA, or CA; the GC ratio of the siRNA molecule preferably should be 45-55%; the siRNA molecule preferably should not contain three of the same nucleotides in a row; the siRNA molecule preferably should not contain seven mixed G/Cs in a row; the siRNA molecule preferably should comprise two nucleotide overhangs (preferably TT) at each 3′ terminus; the target segment preferably should be in the ORF region of the target mRNA and preferably should be at least 75 bp after the initiation ATG and at least 75 bp before the stop codon; and the target segment preferably should not contain more than 16-17 contiguous base pairs of homology to other coding sequences.

Based on some or all of these criteria, preferred phospholamban siRNA target sequences have been identified in human phospholamban mRNA (Genbank Acc. No. NM_{—}002667), mouse phospholamban mRNA (Genbank Acc. No. NM_{—}023129) and rat phospholamban mRNA (Genbank Acc. No. NM_{—}022707) and are set forth in SEQ ID NOs: 1-9, 10-11 and 12-13, respectively. Other siRNA molecules targeted to phospholamban can be designed by one of skill in the art using the aforementioned criteria or other known criteria (e.g., Gilmore et al., J. Drug Targeting 12:315 (2004); Reynolds et al., Nature Biotechnol. 22:326 (2004); Ui-Tei et al., Nucleic Acids Res. 32:936 (2004)). Such criteria are available in various web-based program formats useful for designing and optimizing siRNA molecules (e.g., siDESIGN Center at Dharmacon; BLOCK-iT RNAi Designer at Invitrogen; siRNA Selector at Wistar Insitute; siRNA Selection Program at Whitehead Institute; siRNA Design at Integrated DNA Technologies; siRNA Target Finder at Ambion; and siRNA Target Finder at Genscript).

siRNA molecules targeted to phospholamban can be produced in vitro by annealing two complementary single-stranded RNA molecules together (one of which matches at least a portion of a phospholamban nucleic acid sequence) (e.g., U.S. Pat. No. 6,506,559) or through the use of a short hairpin RNA (shRNA) molecule which folds back on itself to produce the requisite double-stranded portion (Yu et al., Proc. Natl. Acad. Sci. USA 99:6047 (2002)). Such single-stranded RNA molecules can be chemically synthesized (e.g., Elbashir et al., Nature 411:494 (2001)) or produced by in vitro transcription using DNA templates (e.g., Yu et al., Proc. Natl. Acad. Sci. USA 99:6047 (2002)). When chemically synthesized, chemical modifications can be introduced into the siRNA molecules to improve biological stability. Such modifications include phosphorothioate linkages, fluorine-derivatized nucleotides, deoxynucleotide overhangs, 2′-O-methylation, 2′-O-allylation, and locked nucleic acid (LNA) substitutions (Dorset and Tuschl, Nat. Rev. Drug Discov. 3:318 (2004); Gilmore et al., J. Drug Targeting 12:315 (2004)).

siRNA molecules targeted to phospholamban can be introduced into cells to inhibit phospholamban expression. Accordingly, another aspect of the present invention provides a method for inhibiting expression of phospholamban in a cell comprising introducing into a cell at least one siRNA molecule that corresponds to at least a portion of a phospholamban nucleic acid sequence. Although any cell can be targeted, the cell into which the siRNA molecules are introduced is preferably a heart cell, more preferably a cardiomyocyte. In some embodiments, the heart cell is from a patient suffering from heart failure, preferably a human patient.

The siRNA molecules produced herein can be introduced into cells in vitro or ex vivo using techniques well-known in the art, including electroporation, calcium phosphate co-precipitation, microinjection, lipofection, polyfection, and conjugation to cell penetrating peptides (CPPs). The siRNA molecules can also be introduced into cells in vivo by direct delivery into specific organs such as the liver, brain, eye, lung and heart, or systemic delivery into the blood stream or nasal passage using naked siRNA molecules or siRNA molecules encapsulated in biodegradable polymer microspheres (Gilmore et al., J. Drug Targeting 12:315 (2004)).

Alternatively, siRNA molecules targeted to phospholamban can be introduced into cells in vivo by endogenous production from an expression vector(s) encoding the sense and antisense siRNA sequences. Accordingly, another aspect of the present invention provides an expression vector comprising at least one DNA sequence encoding a siRNA molecule corresponding to at least a portion of a phospholamban nucleic acid sequence capable of inhibiting expression of phospholamban in a cell operably linked to a genetic control element capable of directing expression of the siRNA molecule in a cell. Expression vectors can be transfected into cells using any of the methods described above.

Genetic control elements include a transcriptional promoter, and may also include transcription enhancers to elevate the level of mRNA expression, a sequence that encodes a suitable ribosome binding site, and sequences that terminate transcription. Suitable eukaryotic promoters include constitutive RNA polymerase II promoters (e.g., cytomegalovirus (CMV) promoter, the SV40 early promoter region, the promoter contained in the 3′ long terminal repeat of Rous sarcoma virus (RSV), the herpes thymidine kinase (TK) promoter, and the chicken beta-actin promoter), cardiac-tissue-specific RNA polymerase II promoters (e.g., the ventricular myosin light chain 2 (MLC-2v) promoter, and the sodium-calcium exchanger gene H1 promoter (NCX1H1)), and RNA polymerase III promoters (e.g., U6, H1, 7SK and 7SL).

In some embodiments, the sense and antisense strands of siRNA molecules are encoded by different expression vectors (i.e., cotransfected) (e.g., Yu et al., Proc. Natl. Acad. Sci. USA 99:6047 (2002). In other embodiments, the sense and antisense strands of siRNA molecules are encoded by the same expression vector. The sense and antisense strands can be expressed separately from a single expression vector, using either convergent or divergent transcription (e.g., Wang et al., Proc. Natl. Acad. Sci. USA 100:5103 (2003); Tran et al., BMC Biotechnol. 3:21 (2003)). Alternatively, the sense and antisense strands can be expressed together from a single expression vector in the form of a single hairpin RNA molecule, either as a short hairpin RNA (shRNA) molecule (e.g., Arts et al., Genome Res. 13:2325 (2003)) or a long hairpin RNA molecule (e.g., Paddison et al., Proc. Natl. Acad. Sci. USA 99:1443 (2002)).

Although numerous expression vectors can be used to express siRNA molecules in cells (Dorsett and Tuschl, Nat. Rev. Drug Discov. 3:318 (2004)), viral expression vectors are preferred, particularly those that efficiently transduce heart cells (e.g., alphaviral, lentiviral, retroviral, adenoviral, adeno-associated viral (AAV)) (Williams and Koch, Annu. Rev. Physiol. 66:49 (2004); del Monte and Hajjar, J. Physiol. 546.1:49 (2003). Both adenoviral and AAV vectors have been shown to be effective at delivering transgenes (including transgenes directed to phospholamban) into heart cells, including failing cardiomycoytes (e.g., Iwanaga et al., J. Clin. Invest. 113:727 (2004); Seth et al., Proc. Natl. Acad. Sci. USA 101:16683 (2004); Champion et al., Circulation 108:2790 (2003); Li et al., Gene Ther. 10:1807 (2003); Vassalli et al., Int. J. Cardiol. 90:229 (2003); del Monte et al., Circulation 105:904 (2002); Hoshijima et al., Nat. Med. 8:864 (2002); Eizema et al., Circulation 101:2193 (2000); Miyamoto et al., Proc. Natl. Acad. Sci. USA 97:793 (2000); He et al., Circulation 100:974 (1999). Recent reports have demonstrated the use of AAV vectors for sustained gene expression in mouse and hamster myocardium and arteries for over one year (Li et al., Gene Ther. 10:1807 (2003); Vassalli et al., Int. J. Cardiol. 90:229 (2003)). In particular, expression vectors based on AAV serotype 6 have been shown to efficiently transduce both skeletal and cardiac muscle (e.g., Blankinship et al., Mol. Ther. 10:671 (2004)). The present invention also provides for the use of coxsackie viral vectors for delivery of phospholamban siRNA.

Following introduction of the phospholamban siRNA molecules into cells, changes in phospholamban gene product levels can be measured if desired. Phospholamban gene products include, for example, phospholamban mRNA and phospholamban polypeptide, and both can be measured using methods well-known to those skilled in the art. For example, phospholamban mRNA can be directly detected and quantified using, e.g., Northern hybridization, in situ hybridization, dot and slot blots, or oligonucleotide arrays, or can be amplified before detection and quantitation using, e.g., polymerase chain reaction (PCR), reverse-transcription-PCR (RT-PCR), PCR-enzyme-linked immunosorbent assay (PCR-ELISA), or ligase chain reaction (LCR).

Phospholamban polypeptide (or fragments thereof) can be detected and quantified using various well-known immunological assays, such as, e.g., enzyme-linked immunosorbent assay (ELISA), radioimmunoassay (RIA), immunoprecipitation, immunofluorescence, and Western blotting. Anti-phospholamban antibodies (preferably anti-human phospholamban) for use in immunological assays are commercially available from, e.g., EMD Biosciences (San Diego, Calif.), Upstate (Charlottesville, Va.), Abcam (Cambridge, Mass.), Affinity Bioreagents (Golden, Colo.) and Novus Biologicals (Littleton, Colo.), or may be produced by methods well-known to those skilled in the art.

The use of siRNA molecules to inhibit cellular expression of phospholamban finds utilities as methods for the treatment of heart failure. Accordingly, another aspect of the present invention provides a method for treating a patient suffering from heart failure comprising introducing into said patient at least one siRNA molecule that corresponds to at least a portion of a phospholamban nucleic acid sequence. Such a method for treatment of heart failure can be performed using systems that provide for the delivery of siRNA molecules targeted to phospholamban to the heart. Accordingly, another aspect of the present invention provides a system for treating a patient suffering from heart failure comprising at least one siRNA molecule that corresponds to at least a portion of a phospholamban nucleic acid sequence and a means for introducing the siRNA molecule into the heart of the patient. In preferred embodiments, the patient is human.

Numerous well-known methods exist for cardiac gene delivery, including simple direct myocardial injection (Williams and Koch, Annu. Rev. Physiol. 66:49 (2004)). However, because heart failure is a diffuse disease of the heart, a system capable of introducing the siRNA molecules into a large portion of the myocardium rather than a just localized region is preferred. For this purpose, a percutaneous intracoronary delivery system is preferred to gain access to the majority of the myocardial tissue.

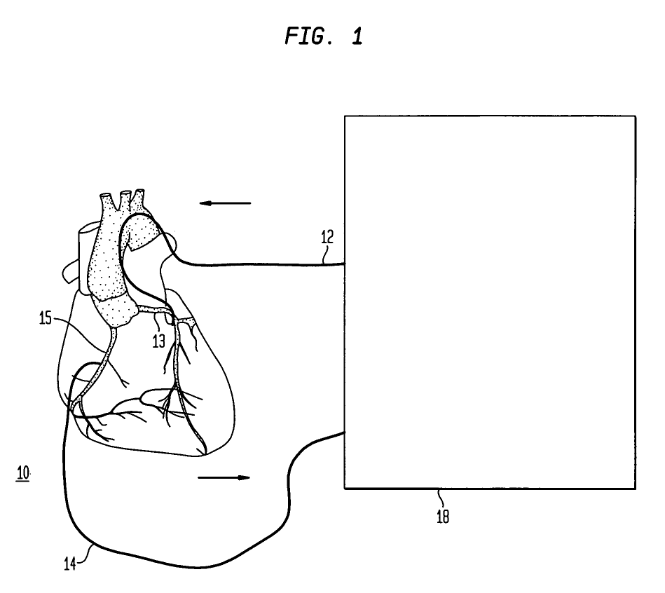

Referring now to FIG. 1, an example of such a system 10 according to one embodiment is shown. For example, two balloon catheters 12 and 14 (such as those disclosed in, e.g., U.S. Patent Application Publication No. U.S. 2004/0116878, and U.S. Pat. Nos. 6,800,065 and 5,938,582) can be advanced into the coronary vasculature of a patient suffering from or at risk for heart failure. Catheter 12 is placed into the left coronary artery 13 or one of its main branches (such as LAD or circumflex artery), and catheter 14 is placed into the coronary sinus 15. Balloons on both catheters are inflated to block the natural flow of the blood through the blocked region. For example, a perfusate is circulated within the coronary vasculature using a pump/reservoir system 18 (e.g., AFFINITY® CVR Cardiotomy/Venous Reservoir, Medtronic, Minneapolis, Minn.) or other suitable device (e.g., Bio-Pump® Plus Centrifugal Blood Pump, Medtronic, Minneapolis, Minn.). The siRNA molecules targeted to phospholamban (preferably delivered in the form of a viral expression vector), which may be placed in solution prior to delivery, are added to the perfusate and circulated through the coronary vasculature to deliver the siRNA molecules to the cardiac tissue. The perfusate can be derived from the subject's own blood or it can be a serum product, or any other appropriate fluid. To prevent ischemia, the perfusate should be balanced for ions of importance and provide O₂ while removing CO₂ and waste products.

Optionally, to enhance the delivery of the siRNA molecules into the cardiac tissue, the pressure in the vasculature may be increased. This may be accomplished by increasing the forward pressure applied by the fluid coming from the catheter in the left coronary artery and/or the back pressure provided by the catheter in the coronary sinus using a pump or other suitable device.

Following a short period of siRNA molecule delivery (e.g., between about 1 minute and 30 minutes, preferably about minutes when AAV is used as the delivery vector and about 9 minutes when adenovirus is used as the delivery vector (Champion et al., Circulation 108:2790 (2003)), the balloon in the left coronary artery is deflated and the catheter is removed, admitting the blood flow from the aorta into the coronary circulation. Negative pressure from the catheter in the coronary sinus maintains the perfusion of the ventricular wall while keeping the venous return containing residual vector out of the patient's systemic circulation. A few minutes later, the balloon in the coronary sinus is also deflated, and the catheter is removed, returning the coronary circulation to the same state as it was before the intervention. To prevent unforeseen toxic effects of phospholamban inhibition, therapy can be titrated by starting with a low dose therapy and assessment of the results, followed by additional deliveries as needed.

Inhibition of phospholamban expression and/or function within the myocardiocytes results in less inhibition of SERCA2 function, leading to an increased ratio of SERCA2 to phospholamban and improved Ca²⁺ re-sequestration by the SR within the heart. The improved Ca²⁺ cycling results in lower Ca²⁺ concentrations during diastole (producing better relaxation and diastolic function) and higher Ca²⁺ release during systole (producing stronger contractions and improved systolic function) (del Monte et al., Circulation 105: 904 (2002); Hoshijima et al., Nat. Med. 8:864 (2002); He et al., Circulation 100:974 (1999)). This improved cardiac function is reflected in various echocardiographic measurements of left ventricular function, including, e.g., higher ejection fraction, reduced systolic wall stress, increased maximum +dP/dt, enhanced minimum +dP/dt, increased −dP/dt, shortened τ, and lower E/A ratio (Iwanaga et al., J. Clin. Invest. 113:727 (2004)).

The reagents, methods and systems of the present invention are also useful for applications in organs other than the heart.

Specific embodiments according to the methods of the present invention will now be described in the following examples. Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the following claims.

### EXAMPLES

### Example 1

Target Sequences for Phospholamban siRNA

The following phospholamban siRNA target sequences were identified based on the open reading frames of human phospholamban mRNA (Genbank Acc. No. NM_{—}002667), mouse phospholamban mRNA (Genbank Acc. No. NM_{—}023129) and rat phospholamban mRNA (Genbank Acc. No. NM_{—}022707). The target sequences were chosen following a BLAST search of sequences within a 40-55% GC range to ensure that only phospholamban mRNA would be targeted.

Human Phospholamban Target Sequence 1:

| | |
|---|---|
| 5′-AAGTCCAATACCTCACTCGCT-3′ | (SEQ ID NO: 1) |

Human Phospholamban Target Sequence 2:

| | |
|---|---|
| 5′-AAGCACGTCAAAAGCTACAGA-3′ | (SEQ ID NO: 2) |

Human Phospholamban Target Sequence 3:

| | |
|---|---|
| 5′-AATTTCTGTCTCATCTTAA-3′ | (SEQ ID NO: 3) |

Human Phospholamban Target Sequence 4:

| | |
|---|---|
| 5′-GGTCTTCACCAAGTATCAA-3′ | (SEQ ID NO: 4) |

Human Phospholamban Target Sequence 5:

| | |
|---|---|
| 5′-GGCCATACTCTTACATAAT-3′ | (SEQ ID NO: 5) |

Human Phospholamban Target Sequence 6:

| | |
|---|---|
| 5′-GGCAAGGAAAATAAAAGAT-3′ | (SEQ ID NO: 6) |

Human Phospholamban Target Sequence 7:

| | |
|---|---|
| 5′-GCACGTCAAAAGCTACAGA-3′ | (SEQ ID NO: 7) |

Human Phospholamban Target Sequence 8:

| | |
|---|---|
| 5′-GGCACTGTAGTGAATTATC-3′ | (SEQ ID NO: 8) |

Human Phospholamban Target Sequence 9:

| | |
|---|---|
| 5′-GCTAGAGTTACCTAGCTTA-3′ | (SEQ ID NO: 9) |

Mouse Phospholamban Target Sequence 1:

| | |
|---|---|
| 5′-AAAGTGCAATACCTCACTCGC-3′ | (SEQ ID NO: 10) |

Mouse Phospholamban Target Sequence 2:

| | |
|---|---|
| 5′-AATTTCTGCCTCATCTTGATA-3′ | (SEQ ID NO: 11) |

Rat Phospholamban Target Sequence 1:

| | |
|---|---|
| 5′-AAAGTGCAATACCTTACTCGC-3′ | (SEQ ID NO: 12) |

Rat Phospholamban Target Sequence 2:

| | |
|---|---|
| 5′-AATTTCTGTCTCATCTTGATA-3′ | (SEQ ID NO: 13) |

### Example 2

Inhibition of Phospholamban Expression

An siRNA duplex (PB0188) targeting rat phospholamban target sequence 1 (SEQ ID NO: 12) was made by in vitro transcription using the Ambioni Silencer™ siRNA Construction Kit (Ambion, Austin, Tex.) following the manufacturer's instructions and quantified by spectrophotometry. The following oligonucleotides were used for generating the PB0188 siRNA duplex:

| | |
|---|---|
| Oligo PB0188AS: | |
| 5′- | (SEQ ID NO: 14) |
| AAAGTCCAATACCTTACTCGCCCTGTCTC-3′; | |
| and | |
| Oligo PB0188SN: | |
| 5′- | (SEQ ID NO: 15) |
| AAGCGAGTAAGGTATTGGACTCCTGTCTC-3′. | |

The last eight nucleotides at the 3′ end of each of oligonucleotides (CCTGTCTC; SEQ ID NO: 16) are not part of the siRNA sequence targeting rat phospholamban mRNA, but are required as part of the Ambion Silencer™ siRNA Construction Kit.

Cultured H9C2 rat cardiomyocyte cells were transfected with amounts of PB0188 siRNA equivalent to a final concentration of 9.375 nM, 18.75 nM, 37.5 nM, or 75 nM using the TransIT-TKO® transfection reagent (Mirus, Madison, Wis.) following the manufacturer's recommended method. Forty-eight hours later, total RNA was harvested from the transfected cells and the amount of phospholamban mRNA in the cells was determined using RT-PCR. Relative amounts of phospholamban mRNA in treated cells versus untransfected cells were determined by comparing the relative intensity of the corresponding bands of RT-PCR products subjected to agarose gel electrophoresis. The results showed that transfection of the H9C2 rat cardiomyocytes with PBO188 siRNA resulted in reduction of phospholamban mRNA by approximately 10% in cells transfected with 9.375 nM siRNA, by approximately 65% in cells transfected with 18.75 nM siRNA, by approximately 35% in cells transfected with 37.5 nM siRNA, and by approximately 100% in cells transfected with 75 nM siRNA, as compared to untransfected control cells. The lack of a consistent dose-response relationship between the concentration of siRNA and the amount of reduction in phospholamban mRNA measured can be attributed to limitations in the quantification method used. Nevertheless, these data clearly indicate that transfection of cardiomyocyte cells with siRNA targeting phospholamban results in reduction in the amount of phospholamban expressed by the cardiomyocyte cells.

All publications cited in the specification, both patent publications and non-patent publications, are indicative of the level of skill of those skilled in the art to which this invention pertains. All these publications are herein fully incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the following claims.

What is claimed:

1. A siRNA molecule corresponding to at least a portion of a phospholamban nucleic acid sequence capable of inhibiting expression of phospholamban in a cell.

2. The siRNA molecule of claim 1, wherein the siRNA molecule comprises a double-stranded structure comprising a sense strand and an antisense strand, wherein said antisense strand comprises a nucleotide sequence that is complementary to at least a portion of a phospholamban nucleic acid sequence and said sense strand comprises a nucleotide sequence that is complementary to at least a portion of the nucleotide sequence of said antisense region, and wherein said sense strand and said antisense strand each comprise about 19-29 nucleotides.

3. An expression vector comprising at least one DNA sequence encoding a siRNA molecule corresponding to at least a portion of a phospholamban nucleic acid sequence capable of inhibiting expression of phospholamban in a cell operably linked to a genetic control element capable of directing expression of said siRNA molecule in a host cell.

4. The expression vector of claim 3, wherein the siRNA molecule is expressed in the form of hairpin RNA molecule.

5. The expression vector of claim 3, wherein the vector is a viral vector.

6. The expression vector of claim 5, wherein the viral vector is an adenoviral vector.

7. The expression vector of claim 5, wherein the viral vector is an adeno-associated viral vector.

8. The method of claim 5, wherein the viral vector is a coxsackie viral vector.

9. A method for inhibiting expression of phospholamban in a heart cell comprising introducing into said heart cell at least one siRNA molecule that corresponds to at least a portion of a phospholamban nucleic acid sequence.

10. The method of claim 9, wherein the siRNA molecule is introduced by expression from an expression vector.

11. The method of claim 10, wherein the expression vector is a viral vector.

12. The method of claim 11, wherein the viral vector is an adenoviral vector.

13. The method of claim 11, wherein the viral vector is an adeno-associated viral vector.

14. The method of claim 11, wherein the viral vector is a coxsackie viral vector.

15. The method of claim 9, wherein the heart cell is a cardiomyocyte.

16. The method of claim 15, wherein the cardiomyocyte is from a patient suffering from heart failure.

17. The method of claim 16, wherein the patient is human.

18. The method of claim 15, wherein the siRNA molecule is introduced in vitro.

19. The method of claim 15, wherein the siRNA molecule is introduced in vivo.

20. The method of claim 19, wherein the siRNA molecule is introduced into the heart.

21. The method of claim 20, wherein the siRNA molecule is introduced into the coronary vasculature.

22. The method of claim 21, wherein the siRNA molecule is introduced into the left coronary artery.

23. The method of claim 22, wherein the siRNA molecule is introduced as a perfusate using a balloon catheter.

24. The method of claim 23, wherein the perfusate comprises oxygen and nutrients.

25. The method of claim 24, further comprising placing a balloon catheter into the coronary sinus.

26. The method of claim 25, further comprising increasing the pressure within the perfused vasculature during introduction of the siRNA molecule.

27. A method for treating a patient suffering from heart failure comprising introducing into said subject at least one siRNA molecule that corresponds to at least a portion of a phospholamban nucleic acid sequence.

28. The method of claim 27, wherein the siRNA molecule is introduced by expression from a viral vector.

29. The method of claim 28, wherein the viral vector is an adenoviral vector.

30. The method of claim 29, wherein the viral vector is an adeno-associated viral vector.

31. The method of claim 28, wherein the viral vector is a coxsackie viral vector.

32. The method of claim 27, wherein the patient is human.

33. The method of claim 27, wherein the siRNA molecule is introduced into the heart.

34. The method of claim 33, wherein the siRNA molecule is introduced into the coronary vasculature.

35. The method of claim 34, wherein the siRNA molecule is introduced into the left coronary artery.

36. The method of claim 35, wherein the siRNA molecule is introduced as a perfusate using a balloon catheter.

37. The method of claim 36, wherein the perfusate comprises oxygen and nutrients.

38. The method of claim 37, further comprising placing a balloon catheter into the coronary sinus.

39. The method of claim 38, further comprising increasing the pressure within the perfused vasculature during introduction of the siRNA molecule.

40. A system for treating a patient suffering from heart failure comprising at least one siRNA molecule that corresponds to at least a portion of a phospholamban nucleic acid sequence and a means for introducing said siRNA molecule to the heart of the patient.

41. The system of claim 40, wherein the means comprises a pump and a catheter.

42. The method of claim 41, wherein the siRNA molecule is introduced by expression from a viral vector.

43. The method of claim 42, wherein the viral vector is an adenoviral vector.

44. The method of claim 42, wherein the viral vector is an adeno-associated viral vector.

45. The method of claim 42, wherein the viral vector is a coxsackie viral vector.

46. The method of claim 41, wherein the patient is human.
